(19) United States
(12) Patent Application Publication
(10) Pub. No.: US 2009/0175788 A1
(43) Pub. Date: Jul. 9, 2009

(54) RADIOLABELLED LIGAND FOR THE GLYCINE 1 TRANSPORTER

(76) Inventors: Gabriella Gentile, Verona (IT); Hugh Jonathan Herdon, Essex (GB); Johannes Passchier, London (GB); Roderick Alan Porter, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/304,136

(22) PCT Filed: Jun. 20, 2007
(86) PCT No.: PCT/EP2007/056117

§ 371 Date: Dec. 10, 2008

(30) Foreign Application Priority Data
Jun. 22, 2006 (GB) 0612435.8

(51) Int. Cl. A61K 51/04; C07C 233/79

(52) U.S. Cl. 424/1.81; 564/185

(57) ABSTRACT

Compounds of formula (I) and salts and solvates thereof, which are radiolabelled ligands for the glycine 1 transporter, are provided:

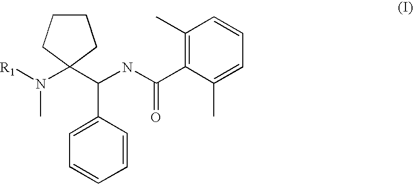

wherein R₁ is ³H, or a C₁₋₆alkyl group incorporating a radionuclide selected from the group consisting of ³H, ¹¹C, ¹⁴C, ¹³N, ¹⁵O, ⁷⁶Br, ¹⁸F, ¹²³I, ¹²⁵I, ¹³¹I, ⁷⁵Br, ⁷⁶Br, ⁷⁷Br and ⁸²Br. Use of the compounds for the labelling and diagnostic imaging of the glycine 1 transporter functionality is disclosed.

The present invention relates to a radiolabelled ligand for the glycine 1 transporter, useful for the labelling and diagnostic imaging of the glycine 1 transporter functionality.

As well as being a major inhibitory neurotransmitter in caudal CNS regions through post synaptic glycine receptors, glycine is also an important excitatory neurotransmitter for glutamatergic neurotransmission through its action as a co-agonist with D-serine at the N-methyl-D-aspartate receptors (NMDAR). Extracellular concentrations of glycine are regulated by the glycine transporters (GlyT1 and GlyT2). These transporters are members of the Na+/Cl− dependent transporter family and mediate the uptake of glycine from the extracellular space into the cytosol. GlyT1 inhibition is currently under review for a number of pathological indications, notably schizophrenia where NMDAR hypofunction is believed to play a major role. This is exemplified by the fact that very similar symptoms to those displayed by schizophrenia patients (e.g. enhanced motor activity, cognitive deficits and increased stereotyped behaviour) can be induced by NMDAR inhibitors such as phenylcyclidine (PCP). These symptoms can be reversed by inhibition of GlyT1 as this leads to increased levels of glycine in the synapse and therefore improved NMDAR neurotransmission. Numerous efforts are being made to develop suitable drug candidates for GlyT1 inhibition and many have recently entered early phase clinical trials in man (V. Eulenburg, W. Armsen, H. Betz, J. Gomeza. Trends Biochem Sci. 2005, 30(6):325-33; H. Betz, J. Gomeza, W. Armsen, P. Scholze, V. Eulenburg. Biochem Soc Trans. 2006, 34(Pt 1), 55-8; D. Javitt. Curr Opin Psychiatry. 2006, 19(2):151-7). Examples of recent compounds developed for inhibition of the type-1 glycine transporter can be found, for example, in L. G. Harsing Jr., Glycine transporter type-1 and its inhibitors. Curr Med Chem. 2006, 13(9), 1017-44; and published international patent applications WO03/055478 (SmithKline Beecham) and WO2006/067423. For example, WO2006/067423 discloses N-[[1-(dimethylamino)cyclopentyl](phenyl)methyl]-2,6-dimethyl benzamide.

Noninvasive, nuclear imaging techniques can be used to obtain basic and diagnostic information about the physiology and biochemistry of living subjects, including experimental animals, patients and volunteers. These techniques rely on the use of imaging instruments that can detect radiation emitted from radiotracers administered to living subjects. The information obtained can be reconstructed to provide planar and tomographic images which reveal the distribution and/or concentration of the radiotracer as a function of time.

Positron emission tomography (PET) is a noninvasive imaging technique that offers the highest spatial and temporal resolution of all nuclear medicine imaging modalities and has the added advantage that it can allow for true quantitation of tracer concentrations in tissues. The technique involves the use of radiotracers, labelled with positron emitting radionuclides, that are designed to have in vivo properties which permit measurement of parameters regarding the physiology or biochemistry of a variety of processes in living tissue (see for example J. Passchier, A. Gee, A. Willemsen, W. Vaalburg, A. van Waarde. Measuring drug-related receptor occupancy with positron emission tomography. Methods. 2002, 27(3), 278-86; and V. J. Cunningham, R. N. Gunn, J. C. Matthews. Quantification in positron emission tomography for research in pharmacology and drug development. Nucl Med Commun. 2004, 25(7), 643-6.)

Compounds can be labelled with positron or gamma emitting radionuclides. The most commonly used positron emitting radionuclides are ¹⁵O, ¹³N, ¹¹C and ¹⁸F, which are accelerator produced and have half lives of 2, 10, 20 and 110 minutes respectively. The most widely used gamma emitting radionuclides are ¹⁸F, ^{99m}Tc, ²⁰¹TI and ¹²³I.

To date, no successful radiolabelled compounds which may be used for, for example, PET or for or SPECT (single photon emission computed tomography), have been reported.

The present invention provides a compound of formula (I) or a salt or solvate thereof:

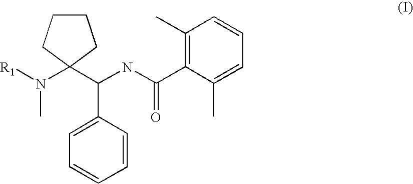

wherein R₁ is a radiolabelled group incorporating or consisting of a radionuclide selected from ³H, ¹¹C, ¹⁴C, ¹³N, ¹⁵O, ⁷⁶Br, ¹⁸F, ¹²³I, ¹²⁵I, ¹³¹I, ⁷⁵Br, ⁷⁶Br, ⁷⁷Br and ⁸²Br.

As used herein, the term “salt” refers to any salt of a compound according to the present invention prepared from an inorganic or organic acid or base, quaternary ammonium salts and internally formed salts. Pharmaceutically acceptable salts are particularly suitable for medical applications because of their greater aqueous solubility relative to the parent compounds. Such salts must clearly have a pharmaceutically acceptable anion or cation. Suitably salts of the compounds of the present invention include acid addition salts formed with inorganic acids such as hydrochloric, hydrobromic, hydroiodic, phosphoric, metaphosphoric, nitric and sulfuric acids, and with organic acids, such as tartaric, acetic, trifluoroacetic, citric, malic, lactic, fumaric, benzoic, formic, propionic, glycolic, gluconic, maleic, succinic, camphorsulfuric, isothionic, mucic, gentisic, isonicotinic, saccharic, glucuronic, furoic, glutamic, ascorbic, anthranilic, salicylic, phenylacetic, mandelic, embonic (pamoic), methanesulfonic, ethanesulfonic, pantothenic, stearic, sulfinilic, alginic, galacturonic and arylsulfonic, for example naphthalene-1,5-disulphonic, naphthalene-1,3-disulphonic, benzenesulfonic, and p-toluenesulfonic, acids; base addition salts formed with alkali metals and alkaline earth metals and organic bases such as N,N-dibenzylethylenediamine, chloroprocaine, choline, diethanolamine, ethylenediamine, meglumaine (N-methylglucamine), lysine and procaine; and internally formed salts. Salts having a non-pharmaceutically acceptable anion or cation are within the scope of the invention as useful intermediates for the preparation of pharmaceutically acceptable salts and/or for use in non-therapeutic, for example, in vitro, situations. The salts may have any suitable stoichiometry. For example, a salt may have 1:1 or 2:1 stoichiometry. Non-integral stoichiometry ratios are also possible.

As used herein, the term “solvate” refers to a complex of variable stoichiometry formed by a solute (in this invention, a compound of formula (I) or a salt thereof) and a solvent. Such solvents for the purpose of the invention may not interfere with the biological activity of the solute. Examples of suitable solvents include, but are not limited to, water, methanol, ethanol and acetic acid. In one embodiment, the solvent used is a pharmaceutically acceptable solvent. Examples of suitable pharmaceutically acceptable solvents include water, ethanol and acetic acid. In one embodiment, the solvent is water.

The compounds of formula (I) have an asymmetric carbon as shown by the arrow below, and thus exist in two enantiomeric forms:

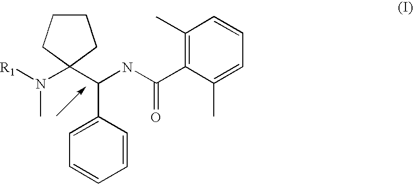

The individual enantiomers and racemic mixtures of these are included within the scope of the present invention.

Thus in one embodiment, the present invention provides R₁-(±)-N-methyl]-N-[[1-(dimethylamino)cyclopentyl](phenyl)methyl]-2,6-dimethylbenzamide, wherein R₁ is a radiolabelled group incorporating or consisting of a radionuclide selected from ³H, ¹¹C, ¹⁴C, ¹³N, ¹⁵O, ⁷⁶Br, ¹⁸F ¹²³I, ¹²⁵I, ¹³¹I, ⁷⁵Br, ⁷⁶Br, ⁷⁷Br and ⁸²Br.

In another embodiment, the present invention provides R₁-(+)-N-methyl]-N-[[1-(dimethylamino)cyclopentyl](phenyl)methyl]-2,6-dimethylbenzamide, wherein R₁ is a radiolabelled group incorporating or consisting of a radionuclide selected from ³H, ¹¹C, ¹⁴C, ¹³N, ¹⁵O, ⁷⁶Br, ¹⁸F, ¹²³I, ¹²⁵I, ¹³¹I, ⁷⁵Br, ⁷⁶Br, ⁷⁷Br and ⁸²Br.

In another embodiment, the present invention provides R₁-(−)-N-methyl]-N-[[1-(dimethylamino)cyclopentyl](phenyl)methyl]-2,6-dimethylbenzamide, wherein R₁ is a radiolabelled group incorporating or consisting of a radionuclide selected from ³H, ¹¹C, ¹⁴C, ¹³N, ¹⁵O, ⁷⁶Br, ¹⁸F, ¹²³I, ¹²⁵I, ¹³¹I, ⁷⁵Br, ⁷⁶Br, ⁷⁷Br and ⁸²Br.

In one embodiment, an optically pure enantiomer is desired. The term “optically pure enantiomer” means that the compound contains greater than about 90% of the desired isomer by weight, such as greater than about 95% of the desired isomer by weight, or greater than about 99% of the desired isomer by weight, said weight percent based upon the total weight of the isomer(s) of the compound. In some cases, one enantiomer of a particular structure may have a significantly higher activity than the other enantiomer of the same structure. Chirally pure, or chirally enriched compounds may be prepared by chirally selective synthesis or by separation of enantiomers. The separation of enantiomers may be carried out on the final product or, alternatively on a suitable intermediate.

It should also be understood that compounds of formula (I) may exist in tautomeric forms other than that shown in the formula and these are also included within the scope of the present invention.

Compounds of formula (I) incorporate a radionuclide selected from: ³H, ¹¹C, ¹⁴C, ¹³N, ¹⁵O, ⁷⁶Br, ¹⁸F, ¹²³I, ¹²⁵I, ¹³¹I, ⁷⁵Br, ⁷⁶Br, ⁷⁷Br and ⁸²Br. The choice of radionuclide will depend on the specific analytical or pharmaceutical application. Therefore, in one embodiment, for in vitro labelling of glycine transporter subtype 1 (GlyT1), and for competition assays, compounds that incorporate ³H, ¹²⁵I or ⁷⁷Br may be used. In one embodiment, for diagnostic and investigative imaging agents, compounds that incorporate ¹¹C, ¹⁸F, ¹²³I or ⁷⁶Br may be used. Incorporation of a chelating radionuclide may be useful in certain applications.

In one embodiment, R₁ is a radionuclide selected from ³H, ¹¹C, ¹⁴C, ¹³N, ¹⁵O, ⁷⁶Br, ¹⁸F, ¹²³I, ¹²⁵I, ¹³¹I, ⁷⁵Br, ⁷⁶Br, ⁷⁷Br and ⁸²Br.

In another embodiment, R₁ is a C₁₋₆alkyl group incorporating a radionuclide selected from ³H, ¹¹C, ¹⁴C, ¹³N, ¹⁵O, ⁷⁶Br, ¹⁸F, ¹²³I, ¹²⁵I, ¹³¹I, ⁷⁵Br, ⁷⁶Br, ⁷⁷Br and ⁸²Br. The term “C₁₋₆alkyl” refers to an alkyl group having from one to six carbon atoms, in all isomeric forms, such as methyl, ethyl, propyl, isopropyl, butyl, isobutyl, sec-butyl, tert-butyl, pentyl, neopentyl, sec-pentyl, n-pentyl, isopentyl, tert-pentyl and hexyl.

In one embodiment, the radionuclide is ¹¹C.

In one embodiment, R₁ is a C₁₋₆alkyl group incorporating a ¹¹C.

In one embodiment, there is provided [¹¹C-(±)-N-methyl]-N-[[1-(dimethylamino)cyclopentyl](phenyl)methyl]-2,6-dimethylbenzamide (hereinafter referred to as “Compound A”) or a salt or solvate thereof.

In one embodiment, there is provided [¹¹C-(+)-N-methyl]-N-[[1-(dimethylamino)cyclopentyl](phenyl)methyl]-2,6-dimethylbenzamide or a salt or solvate thereof.

In one embodiment, there is provided [¹¹C-(−)-N-methyl]-N-[[1-(dimethylamino)cyclopentyl](phenyl)methyl]-2,6-dimethylbenzamide or a salt or solvate thereof.

In one embodiment, R₁ is a C₁₋₆alkyl group incorporating 1, 2 or 3 or more ³H. For example, R₁ is [³H]CH₂; or R₁ is [³H]₂CH; or R₁ is [³H]₃C.

In one embodiment, the radionuclide is ³H.

In one embodiment, there is provided [³H-(±)-N-methyl]-N-[[1-(dimethylamino)cyclopentyl](phenyl)methyl]-2,6-dimethylbenzamide or a salt or solvate thereof.

In one embodiment, there is provided [³H-(+)-N-methyl]-N-[[1-(dimethylamino)cyclopentyl](phenyl)methyl]-2,6-dimethylbenzamide or a salt or solvate thereof.

In one embodiment, there is provided [³H-(−)-N-methyl]-N-[[1-(dimethylamino)cyclopentyl](phenyl)methyl]-2,6-dimethylbenzamide or a salt or solvate thereof.

The compounds of formula (I) may have the ability to crystallise in more than one form. This is a characteristic known as polymorphism, and it is understood that such polymorphic forms (“polymorphs”) are within the scope of formula (I). Polymorphism generally can occur as a response to changes in temperature or pressure or both and can also result from variations in the crystallisation process. Polymorphs can be distinguished by various physical characteristics known in the art such as x-ray diffraction patterns, solubility, and melting point.

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the working Examples.

Scheme 1 represents a synthetic route towards compounds of formula (I) wherein R₁ is a radiolabelled group:

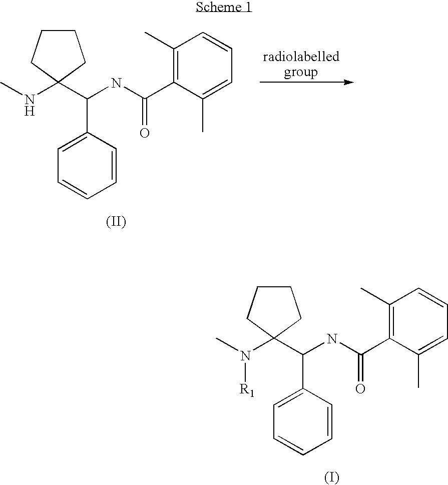

The compound of formula (II) above is 2,6-dimethyl-N-[[1-(methylamino)cyclopentyl](phenyl)methyl] benzamide. A synthetic route for the synthesis of this compound is shown in Scheme 2.

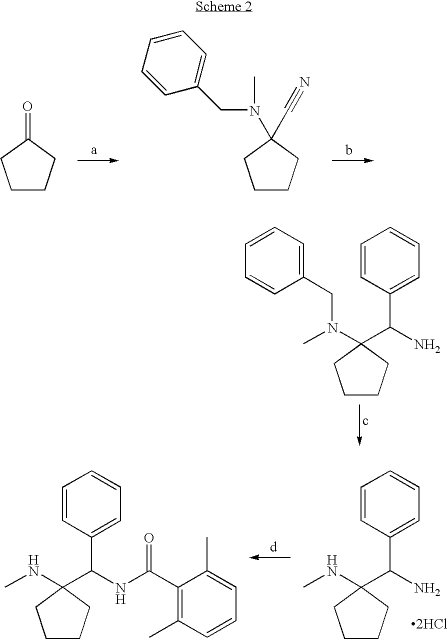

Step a is carried out for example in the presence of inorganic cyanide, for example potassium cyanide, in solvent such as water; or by reaction of the pyrrolidinone with the amine and trimethylsilyl cyanide in either the absence of solvent or in a solvent such as acetic acid. Step b can be achieved by successive reaction with an appropriate organometallic reagent, for example phenyllithium, in a suitable inert solvent for example tetrahydrofuran, followed by reduction with a reducing agent, for example, sodium borohydride in a suitable solvent, for example methanol. Acylation step d can be achieved by reaction with a compound of formula (III):

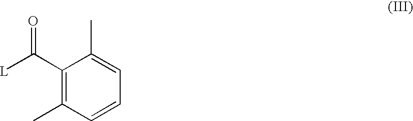

wherein L is a suitable leaving group. Examples of leaving groups include halogen, hydroxy, OC(═O)alkyl, OC(═O)O-alkyl and OSO₂Me. L may be halogen and acylation in step (iii) may be carried out in an inert solvent such as dichloromethane, in the presence of a base such as triethylamine. When L represents hydroxy, the reaction may take place in an inert solvent such as dichloromethane in the presence of a coupling reagent, for example a diimide reagent such as N,N dicyclohexylcarbodiimide (DCC), N-(3-(dimethylamino)propyl)-N-ethylcarbodiimide hydrochloride (EDC), polymer-supported EDC, polymer-supported DCC or O-(7-azabenzotriazol-1-yl)-1,1,3,3-tetramethyluronium hexafluoro phosphate (HATU).

The present invention provides a method for the preparation of a compound of formula (I) or a salt or solvate thereof, comprising reacting 2,6-dimethyl-N-[[1-(methylamino)cyclopentyl](phenyl)methyl] benzamide with a compound of formula (IV):

R₁-X (IV)

wherein R₁ is group containing a radionuclide selected from ³H, ¹¹C, ¹⁴C, ¹³N, ¹⁵O, ⁷⁶Br, ¹⁸F, ¹²³I, ¹²⁵I, ¹³¹I, ⁷⁵Br, ⁷⁶Br, ⁷⁷Br and ⁸²Br, and X is a leaving group; and thereafter optionally forming a salt or solvate thereof. In one embodiment, X is a halogen such as iodine.

In one embodiment, 2,6-dimethyl-N-[[1-(methylamino)cyclopentyl](phenyl)methyl] benzamide is reacted with [¹¹C]methyl iodide to provide [¹¹C—N-methyl]-N-[[1-(dimethylamino)cyclopentyl](phenyl)methyl]-2,6-dimethylbenzamide.

Compounds of formula (I) may be used in pre-clinical studies, for example in GlyT1 binding studies and GlyT1 distribution studies, and in clinical studies, for example to evaluate the role of glycine transporter subtype 1 (GlyT1) in a variety of disease areas where GlyT1 is believed to be involved. They may be used in healthy subjects as well as in those affected by a disease, including a disease which is mediated by GlyT1. For example, they may be used to characterise any differences between healthy subjects and those affected by a disease, which may for example aid the decision process for determining which drug to prescribe.

Thus the present invention provides a compound of formula (I) or a salt or solvate thereof for use in therapy. In one embodiment, the present invention provides a compound of formula (I) or a salt or solvate thereof for use as a GlyT1 ligand. In one embodiment, the present invention provides a compound of formula (I) or a salt or solvate thereof for use in a GlyT1 binding study. In one embodiment, the present invention provides a compound of formula (I) or a salt or solvate thereof for use as a PET ligand or a SPECT ligand.

The present invention provides a method for labelling GlyT1 in a mammal which comprises administering to a mammal an effective amount of a compound of formula (I) or a salt or solvate thereof.

Thus the present invention provides a method for delineation of GlyT1 in a mammal, which comprises administering to a mammal an effective amount of a compound of formula (I) or a salt or solvate thereof.

The present invention also provides a method for diagnostic imaging of GlyT1 which comprises administering to a mammal an effective amount of a compound of formula (I) or a salt or solvate thereof.

The present invention also provides a method for diagnostic imaging of tissues expressing GlyT1 in a mammal which comprises administering to a mammal an effective amount of a compound of formula (I) or a salt or solvate thereof.

The present invention also provides a method for diagnostic imaging of glycine transporter subtype 1 (GlyT1) in the brain of a mammal, which comprises administering to a mammal an effective amount of a compound of formula (I) or a salt or solvate thereof.

The present invention further provides a method for the detection or quantification of GlyT1 functionality in mammalian tissue which comprises administering to a mammal in which such detection or quantification is desired an effective amount of a compound of formula (I) or a salt or solvate thereof.

The present invention also provides use of a compound of formula (I) or a salt or solvate thereof in the manufacture of a composition for labelling GlyT1 in a mammal.

The present invention also provides use of a compound of formula (I) or a salt or solvate thereof in the manufacture of a composition for delineation of GlyT1 in a mammal.

The present invention also provides use of a compound of formula (I) or a salt or solvate thereof in the manufacture of a composition for diagnostic imaging of GlyT1.

The present invention also provides use of a compound of formula (I) or a salt or solvate thereof in the manufacture of a composition for diagnostic imaging of tissues expressing GlyT1 in a mammal.

The present invention also provides use of a compound of formula (I) or a salt or solvate thereof in the manufacture of a composition for diagnostic imaging of glycine transporter subtype 1 (GlyT1) in the brain of a mammal.

The present invention further provides use of a compound of formula (I) or a salt or solvate thereof in the manufacture of a composition for detection or quantification of GlyT1 functionality in mammalian tissue.

In one embodiment, in the above uses and methods of the present invention, the mammal is human. In one embodiment, the human is not affected by a disorder mediated by GlyT1. In one embodiment, the human is affected by a disorder mediated by GlyT1.

As used herein, the terms “a disorder mediated by GlyT1” and “a disease mediated by GlyT1” refer to a disorder or disease that may be treated by the administration of a medicament that alters the activity of the GlyT1 transporter. The action of GlyT1 transporters affects the local concentration of glycine around NMDA receptors. As a certain amount of glycine is needed for the efficient functioning of NMDA receptors, any change to that local concentration can affect NMDA-mediated neurotransmission. Changes in NMDA-mediated neurotransmission have been implicated in certain neuropsychiatric disorders such as dementia, depression and psychoses, for example schizophrenia, and learning and memory disorders, for example attention deficit disorders and autism. Thus, alterations in the activity of the GlyT1 transporter are expected to influence such disorders.

The disorders mediated by GlyT1 referred to herein include neurological and neuropsychiatric disorders, including psychoses such as schizophrenia, dementia and other forms of impaired cognition such as attention deficit disorders and organic brain syndromes. Other neuropsychiatric disorders include drug-induced (phencyclidine, ketamine and other dissociative anesthetics, amphetamine and other psychostimulants and cocaine) psychosis, psychosis associated with affective disorders, brief reactive psychosis, schizoaffective psychosis, and psychosis NOS, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), and NMDA receptor-related disorders such as autism, depression, benign forgetfulness, childhood learning disorders and closed head injury.

Within the context of the present invention, the terms used herein are classified in the Diagnostic and Statistical Manual of Mental Disorders, 4^{th} Edition, published by the American Psychiatric Association (DSM-IV) and/or the International Classification of Diseases, 10^{th} Edition (ICD-10). The various subtypes of the disorders mentioned herein are contemplated as part of the present invention. Numbers in brackets after the listed diseases below refer to the classification code in DSM-IV.

In particular, the compounds of formula (I) may be used in mammals affected by schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-induced Psychotic Disorder including the subtypes With Delusions (293.81) and With Hallucinations (293.82); and Psychotic Disorder Not Otherwise Specified (298.9).

The compounds of formula (I) may be used in mammals affected by mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Mk/ajor Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90).

The compounds of formula (I) are also of use in mammals affected by anxiety disorders including Panic Attack, Agoraphobia, Panic Disorder, Agoraphobia Without History of Panic Disorder (300.22), Specific Phobia (300.29) including the subtypes Animal Type, Natural Environment Type, Blood-Injection-Injury Type, Situational Type and Other Type), Social Phobia (300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-induced Anxiety Disorder and Anxiety Disorder Not Otherwise Specified (300.00).

The compounds of formula (I) may be used in mammals affected by substance-related disorders including Substance Use Disorders such as Substance Dependence and Substance Abuse; Substance-induced Disorders such as Substance Intoxication, Substance Withdrawal, Substance-induced Delirium, Substance-induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-induced Persisting Amnestic Disorder, Alcohol-Induced Psychotic Disorder, Alcohol-induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-induced Anxiety Disorder, Cocaine-induced Sexual Dysfunction, Cocaine-induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide.

The compounds of formula (I) may be used in mammals affected by sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition; and Substance-Induced Sleep Disorder including the subtypes Insomnia Type, Hypersomnia Type, Parasomnia Type and Mixed Type.

The compounds of formula (I) may be used in mammals affected by eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; and Eating Disorder Not Otherwise Specified (307.50).

The compounds of formula (I) may be used in mammals affected by Autistic Disorder (299.00); Attention-Deficit/Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-impulse Type (314.01) and Attention-Deficit/Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23).

The compounds of formula (I) may be used in mammals affected by Personality Disorders including the subtypes Paranoid Personality Disorder (301.0), Schizoid Personality Disorder (301.20), Schizotypal Personality Disorder (301.22), Antisocial Personality Disorder (301.7), Borderline Personality Disorder (301.83), Histrionic Personality Disorder (301.50), Narcissistic Personality Disorder (301.81), Avoidant Personality Disorder (301.82), Dependent Personality Disorder (301.6), Obsessive-Compulsive Personality Disorder (301.4) and Personality Disorder Not Otherwise Specified (301.9).

The compounds of Formula (I) may be used in mammals affected by cognition impairment in other diseases such as schizophrenia, bipolar disorder, depression, other psychiatric disorders and psychotic conditions associated with cognitive impairment. Within the context of the present invention, the term cognitive impairment includes for example the treatment of impairment of cognitive functions including attention, orientation, learning disorders, memory (i.e. memory disorders, amnesia, amnesic disorders, transient global amnesia syndrome and age-associated memory impairment) and language function; cognitive impairment as a result of stroke, Alzheimer's disease, Huntington's disease, Pick disease, Aids-related dementia or other dementia states such as Multiinfarct dementia, alcoholic dementia, hypotiroidism-related dementia, and dementia associated to other degenerative disorders such as cerebellar atrophy and amyotropic lateral sclerosis; other acute or sub-acute conditions that may cause cognitive decline such as delirium or depression (pseudodementia states) trauma, head trauma, age related cognitive decline, stroke, neurodegeneration, drug-induced states, neurotoxic agents, mild cognitive impairment, age related cognitive impairment, autism related cognitive impairment, Down's syndrome, cognitive deficit related to psychosis, and post-electroconvulsive treatment related cognitive disorders; and dyskinetic disorders such as Parkinson's disease, neuroleptic-induced parkinsonism, and tardive dyskinesias.

The compounds of formula (I) may be used in mammals affected by sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9).

The compounds of formula (I) may be used in mammals affected by convulsions, and particularly epilepsy in humans. “Epilepsy” is intended to include the following seizures: simple partial seizures, complex partial seizures, secondary generalised seizures, generalised seizures including absence seizures, myoclonic seizures, clonic seizures, tonic seizures, tonic clonic seizures and atonic seizures.

The compounds of formula (I) may be used in mammals affected by neuropathic pain, for example in diabetic neuropathy, sciatica, non-specific lower back pain, multiple sclerosis pain, fibromyalgia, HIV-related neuropathy, neuralgia such as post-herpetic neuralgia and trigeminal neuralgia and pain resulting from physical trauma, amputation, cancer, toxins or chronic inflammatory conditions.

In a further aspect of the invention, there is provided a pharmaceutical composition comprising a compound of formula (I) as hereinbefore described or a salt or solvate thereof, and at least one pharmaceutically acceptable carrier, diluent or excipient.

The carrier must be pharmaceutically acceptable to the recipient and must be compatible with, i.e. not have a deleterious effect upon, the other ingredients in the composition. The carrier may be a solid or a liquid and may be formulated with at least one compound of formula (I) or a salt or solvate thereof as a unit dose formulation. If desired, other pharmaceutically active ingredients may also be incorporated in the pharmaceutical compositions of the invention.

Possible formulations include those suitable for oral, sub-lingual, buccal, parenteral (for example, subcutaneous, intramuscular, or intravenous), rectal, topical and intranasal administration and in forms suitable for administration by inhalation or insufflation (either through the mouth or nose). In one embodiment, oral administration is provided.

Formulations suitable for oral administration may be provided as discrete units, such as tablets, capsules, cachets, or lozenges, each containing a predetermined amount of the active compound; as powders or granules; as solutions or suspensions in aqueous or non-aqueous liquids; or as oil-in-water or water-in-oil emulsions. For example, a compound of the invention may be prepared as a formulation with a controlled release profile. This may be in any of the above mentioned pharmaceutical forms. For example, it may be a gel formulation in a non aqueous oily vehicle, for example Miglyol, with a suitable gelling agent if required, for example methyl cellulose or hydrophobic colloidal silica.

Formulations suitable for sublingual or buccal administration include lozenges comprising the active compound and, typically, a flavoured base, such as sugar and acacia or tragacanth and pastilles comprising the active compound in an inert base, such as gelatin and glycerin or sucrose and acacia.

Formulations suitable for parenteral administration typically comprise sterile aqueous solutions containing a predetermined concentration of the active compound; the solution may be isotonic with the blood of the intended recipient. Although such solutions may be administered intraveneously, they may also be administered by subcutaneous or intramuscular injection.

Formulations suitable for rectal administration may be provided as unit-dose suppositories comprising the active ingredient and one or more solid carriers forming the suppository base, for example, cocoa butter.

Formulations suitable for topical or intranasal application include ointments, creams, lotions, pastes, gels, sprays, aerosols and oils. Suitable carriers for such formulations include petroleum jelly, lanolin, polyethylene glycols, alcohols, and combinations thereof.

Formulations of compounds of the invention may, for example, be composed so as to improve the exposure profile of the compound of the invention.

Compositions suitable for transdermal administration include ointments, gels and patches. In one embodiment, the composition is in unit dose form such as a tablet, capsule or ampoule.

The formulations of the invention may be prepared by any suitable method, typically by uniformly and intimately admixing the active compound(s) with liquids or finely divided solid carriers, or both, in the required proportions and then, if necessary, shaping the resulting mixture into the desired shape.

For example, a tablet may be prepared by compressing an intimate mixture comprising a powder or granules of the active ingredient and one or more optional ingredients, such as a binder, lubricant, inert diluent, or surface active dispersing agent, or by moulding an intimate mixture of powdered active ingredient and inert liquid diluent.

Aqueous solutions for parenteral administration are typically prepared by dissolving the active compound in sufficient water to give the desired concentration and then rendering the resulting solution sterile and isotonic.

### Abbreviations:

THF tetrahydrofuran
DCM dichloromethane
DMF dimethylformamide
HATU O-(7-azabenzotriazol-1-yl)-N,N,N′,N′-tetramethyluroniumhexa fluorophosphate
EDC N-(3-(dimethylamino)propyl)-N-ethylcarbodiimide hydrochloride
HOAt 3H-(1,2,3)-triazolo(4,5-b)pyridine-3-ol
NMP N-methylpyrrolidinone
DIPEA N,N-diisopropylethylamine
HOBt 1-hydroxybenzotriazole hydrate

### Analytical LC/MS Chromatography Conditions:

### Method A

Column: Waters Atlantis 50 mm×4.6 mm, 3 um particle size
Mobile phase: A: 0.05% Formic acid+Water
B: Acetonitrile+0.05% Formic acid
Gradient: 5-min runtime: 3% B to 97% B over 4 min
Flow rate: 3 ml/min
UV wavelength range: 220-330 nm
Temperature: 30° C.

Throughout the examples section, the following terminology is adopted with regard to chiral compounds: when a mixture of two enantiomers has been prepared, the compound is described as (±); and when a single enantiomer (that is to say mixture chirally enriched in one of the enantiomers) has been prepared, it is referred to as “chiral”.

Where reactions are described as having been carried out in a similar manner to earlier, more completely described reactions, the general reaction conditions used were essentially the same. Work up conditions used were of the types standard in the art, but may have been adapted from one reaction to another.

### Description 1:1-[Methyl(phenylmethyl)amino]cyclopentanecarbonitrile

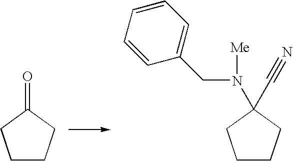

Potassium cyanide (5.41 g; 83 mmol) in water (45 ml) was added dropwise over 10 minutes to a stirred, ice-cooled mixture of cyclopentanone (7 g; 83 mmol) and N-methylbenzylamine (10.08 g; 83 mmol). After stirring for 18 hours at room temperature the mixture was extracted with diethyl ether (2×100 ml). Combined extracts were washed with brine (100 ml), dried (Na₂SO₄) and the solvent removed under reduced pressure to afford 1-[Methyl(phenylmethyl)amino]cyclopentanecarbonitrile. ¹H NMR (CDCl₃) δ: 1.90 (6H, m), 2.20 (3H, s), 2.3 (2H, m), 3.62 (2H, s), 7.25 (1H, m), 7.32 (4H, m); Mass Spectrum (Electrospray LC/MS): Found 188 (MH⁺-HCN). C₁₄H₁₈N₂ requires 214. Ret. time 1.21 min.

### Description 2 (±)-{1-[Amino(phenyl)methyl]cyclopentyl}methyl (phenylmethyl)amine

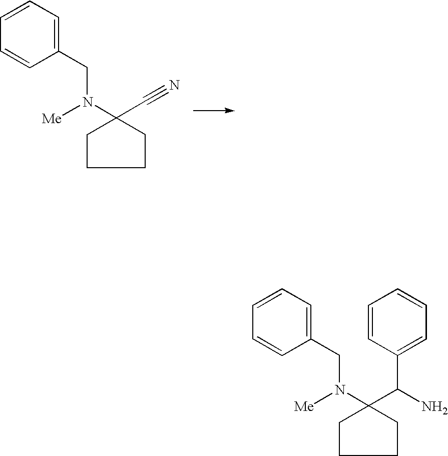

To a stirred solution of 1-[methyl(phenylmethyl)amino]cyclopentanecarbonitrile (D1 (6.0 g; 28 mmol) in THF at −70° C. under argon was added phenyllithium in di-n-butylether (16.21 ml of 1.9M solution; 30.8 mmol) slowly. The reaction mixture was allowed to warm to room temperature over 3 hours before being recooled to 0° C. Methanol (60 ml) was added followed by sodium borohydride (3.2 g, 84 mmol) portionwise. The reaction was stirred overnight at 20° C., cooled to 0° C. and saturated sodium hydrogen carbonate added. The mixture was extracted with ethyl acetate (2×50 ml), the combined extracts dried (Na₂SO₄) and the solvent evaporated. Chromatography on silica eluting with 0-10% methanol in dichloromethane gradient gave the title compound (3.90 g, 47%). Mass Spectrum (Electrospray LC/MS), API⁺: Found 295 (MH⁺). C₂₀H₂₆N₂ requires 294. Ret. time 2.12 min.

### Description 3 (±)-{1-[Amino(phenyl)methyl]cyclopentyl}methylamine Dihydrochloride

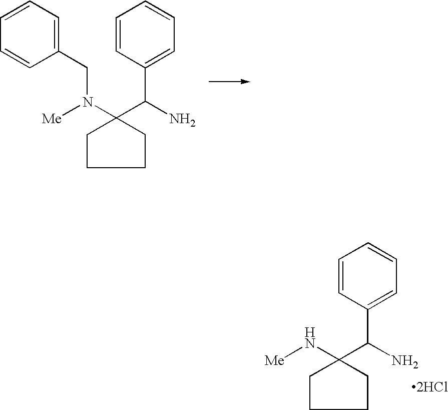

To a solution of (±)-{1-[amino(phenyl)methyl]cyclopentyl}methyl(phenylmethyl)amine (D2 (0.5 g; 1.7 mmol) in ethanol was added 3N HCl (1 ml) and 10% palladium on carbon (0.1 g). The catalytic hydrogenation was carried out for 16 h at room temperature and atmospheric pressure. The catalyst was filtered off through kieselguhr and the filtrate evaporated under reduced pressure to give the title compound (0.32 g; 69%). ¹H NMR (DMSO) δ: 1.3-2.2 (8H, m), 2.5 (3H, s), 4.6 (1H, s), 7.4 (3H, m), 7.6 (2H, m), 8.0 (2H, bs), 9.0 (1H, bs).

### Description 4 {1-[Amino(phenyl)methyl]cyclopentyl}methylamine

### Enantiomer 1 and Enantiomer 2

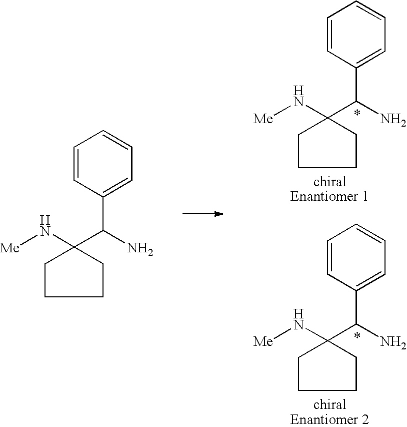

Racemic (±)-{1-[amino(phenyl)methyl]cyclopentyl}methylamine dihydrochloride (D3 (2 g) was partitioned between dichloromethane and 1N sodium hydroxide. The organic layer was washed with brine, dried (Na₂SO₄) and evaporated to afford the corresponding free base (1.435 g). Of this (0.342 g; 1.67 mmol) was separated by preparative chiral HPLC to afford the title products enantiomer 1 (0.134 g); Chiral HPLC: 99.8% ee; ¹H NMR (CDCl₃) δ: 1.30-1.78 (11H, m), 2.33 (3H, s), 4.08 (1H, s), 7.22 (1H, m), 7.28 (2H, m), 7.35 (2H, m), and enantiomer 2 (0.127 g); Chiral HPLC: 99.8% ee; ¹H NMR (CDCl₃) δ: 1.30-1.78 (11H, m), 2.33 (3H, s), 4.08 (1H, s), 7.22 (1H, m), 7.28 (2H, m), 7.35 (2H, m).

### Analytical HPLC Conditions:

Column: Chiral OD 10 micron particle size 20 mm i.d.×250 mm
Mobile phase: Heptane:Absolute Ethanol (90:10 v/v)

### Gradient: Isocratic

### UV Wavelength: 215 nm

Flow rate: 1 ml/min
Ret. Time: 7.5 min (Enantiomer 1); 15.6 min (Enantiomer 2)

### Preparative HPLC Conditions:

Column: Chiral OD 10 micron particle size 20 mm i.d.×250 mm
Mobile phase: Heptane:Absolute Ethanol (90:10 v/v)

### Gradient: Isocratic

### UV Wavelength: 215 nm

Flow rate: 17 ml/min

### Description 5: 2,6-Dimethyl-N-[[1-(methylamino)cyclopentyl](phenyl)methyl]

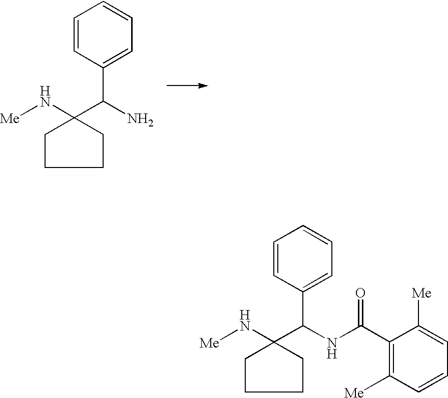

### Benzamide Chiral

To a solution of 2,6-dimethylbenzoic acid (0.100 g; 0.668 mmol) in DMF (5 ml) and DIPEA (0.12 ml) was added {[amino(phenyl)methyl]cyclopentyl} methylamine (D4) enantiomer 2 (0.124 g; 0.608 mmol) and HATU (0.254 g; 0.668 mmol). The resulting mixture was allowed to stir at room temperature for 3 days and then the DMF was evaporated off under reduced pressure. Residual material was partitioned between ethyl acetate and water, washed with water and the organic layer was dried (Na₂SO₄) and evaporated. The residual material was dissolved in DCM (2 ml) and loaded onto an SCX cartridge. Washing with DCM, then methanol followed by elution with 1M ammonia in methanol afforded the title product (155 mg; 76%). ¹H NMR (CDCl3) δ: 1.3-1.8 (9H, m), 2.21 (3H, s), 2.28 (6H, s), 5.07 (1H, m), 7.0 (2H, m), 7.1-7.4 (7H, m). Mass Spectrum (Electrospray LC/MS). Found 337 (MH⁺). C₂₂H₂₈N₂O requires 336. Ret. time: 1.86 min.

### EXAMPLE 1

### [¹¹C—N-methyl]-N-[[1-(dimethylamino)cyclopentyl](phenyl)methyl]-2,6-dimethylbenzamide (Compound A)

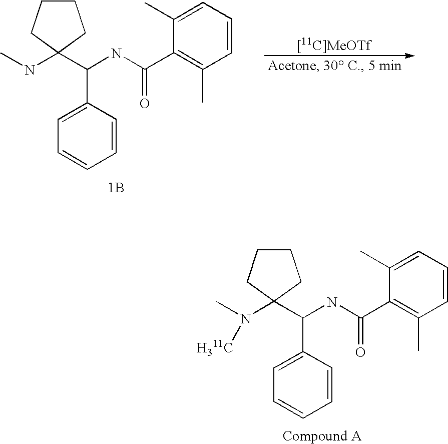

Compound A was prepared by N-alkylation of the benzamide precursor 1B using cyclotron-produced [¹¹C]methyl iodide. Carbon-11 was produced as [¹¹C]CO₂ by bombarding nitrogen with 16.5 MeV protons according to the ¹⁴N(p,α)¹¹C reaction, the presence of a small amount of oxygen (0.5%) in the target gas converting the ¹¹C into [¹¹C]CO₂. Subsequently, [¹¹C]CO₂ was converted into [¹¹C]Mel by catalytic reduction (Ni) which gives the [¹¹C]CH₄ intermediate followed by gas phase iodination with iodine. The [¹¹C]methyl iodide was converted on-line to [¹¹C]MeOTf by passing it through a heated quartz tube (200° C.) filled with AgOTf. The [¹¹C]MeOTf was subsequently delivered to the reaction vial containing the precursor 1B acetone at room temperature. The reaction mix was heated to 30° C. for 5 min. Following a 70 min irradiation, typical syntheses provide 0.6 to 3.5 GBq of Compound A. For all the productions, the radiochemical purity was greater than 99% and the specific activity ranged from 24 to 1000 GBq/umol. The average total synthesis time including HPLC purification and formulation was approximately 40 min from the end of bombardment.

The precursor (1.0 mg) dissolved in acetone (300 uL) was placed in a 1 mL glass vial. The [¹¹C]CH₃OTf was delivered as a gas to the reaction vial and bubbled through the solution containing the precursor at room temperature. After delivery of [¹¹C]CH₃I, the sealed vessel was heated to 30° C. for 5 min and injected onto the semi-prep HPLC column (Sphereclone ODS(2) C-18 250×10 mm). HPLC purification was performed at a 8 mL/min flow rate with a mobile phase consisting of acetonitrile and a solution of sodium dihydrogen phosphate (70 mM) (40:60). The product fraction collected after approximately 7.7 min was evaporated to dryness and reformulated in 9 mL 0.9% NaCl and 0.2 mL ethanol. Quality control was performed using analytical HPLC on a Sphereclone ODS(2) C-18 250×4.6 mm using acetonitrile and a solution of sodium dihydrogen phosphate (70 mM) (70:30) as mobile phase at a flow rate of 1 ml/min.

### EXAMPLE 2

### [³H—N-methyl]-N-[[1-(dimethylamino)cyclopentyl](phenyl)methyl]-2,6-dimethylbenzamide (Compound B)

A solution of [³H]methyl nosylate in ethyl acetate (1200 mCi at 33 mCi/ml) was rotary evaporated to dryness and redissolved in ethyl acetate (2 ml). 2,6-Dimethyl-N-[[1-(methylamino)cyclopentyl](phenyl)methyl] benzamide chiral (7 mg) dissolved in ethyl acetate (0.5 ml) containing pentamethylpiperidine (2 μl) was added. The solution was stirred and heated at 50° C. for 18 hours. Labile activity was removed by repeated rotary evaporations with ethanol (3×5 ml). The residue was dissolved in ethanol (25 ml). A portion of the crude product was purified on a Beckman Ultrasphere ODS column, eluting with a water to acetonitrile gradient containing trifluoroacetic acid. The collected product was still found to contain any impurity and was therefore purified again using the same column and eluents but a different gradient. The title compound was collected, rotary evaporated to dryness and the residue dissolved in ethanol.

### Biological Data

### 1. In Vivo Imaging

1.1 PET Imaging with Compound A

The animals (pig, Yorkshire/Danish Landrace (˜40 Kg; n=2) were housed singly in thermostatically controlled (20° C.) and naturally illuminated stalls. They were scanned under terminal anaesthesia (ketamine induced isoflurane anaesthesia) on different days. The left femoral artery and vein of each animal were surgically cannulated using catheters (Avanti® size 4F-7F). Blood samples were collected from the femoral artery and the radiolabelled and non-labelled agents were injected into the femoral vein. Animals were placed supine in a Siemens ECAT EXACT HR tomograph, with the head immobilised in a custom-made holding device. During the study, blood pH, pCO₂ and pO₂ levels were monitored and maintained within the normal physiological range. In addition, BP and heart rate were recorded throughout the study. Compound A was administered intravenously into the femoral vein as a 1 minute bolus injection. PET scanning and arterial blood sampling was commenced upon start of the radioligand administration.

PET images were acquired from 0 to 90 min following administration of Compound A. Compound A readily enters the pig brain; the radioactivity reached its peak uptake at 25 min after administration of the radiotracer and then steadily declined over the remainder of the study. The regional brain distribution of Compound A reflected the known distribution of the glycine transporter subtype 1 (GlyT1) with a higher accumulation in mid-brain, thalamus and cerebellum compared to cortical regions (B. Cubelos, C. Gimenez, F. Zafra., Cereb Cortex. 2005, 15(4), 448-59.)

1.2 PET Imaging with Pharmacological Challenges

In a first study, two sequential high specific activity iv radioligand Compound A administrations were performed in same animal on the same day. Following a baseline scan, the animal was pretreated with a high intravenous dose of the selective glycine transporter subtype 1 (GlyT1) 2-chloro-N—[(S)-[(2S)-1-methyl-2-piperidinyl](phenyl)methyl]-3-(trifluoromethyl)benzamide (0.5 mg/kg), 5 minutes prior to administration of compound A. In a second experiment, an escalating dose of the selective glycine transporter subtype 1 (GlyT1) 2-chloro-N—[(S)-[(2S)-1-methyl-2-piperidinyl](phenyl)methyl]-3-(trifluoromethyl)benzamide (0.001, 0.01 and 0.1 mg/kg) were administered 5 min prior to administration of compound A. [¹⁵O]CO and [¹⁵O]H₂O were administered pre and post administration of unlabelled compound A to provide estimates for cerebral blood volume and to provide anatomical information, respectively. Following administration of the high dose of the selective glycine transporter subtype 1 (GlyT1) 2-chloro-N—[(S)-[(2S)-1-methyl-2-piperidinyl](phenyl)methyl]-3-(trifluoromethyl)benzamide, the specific uptake of radiotracer was blocked leading to a homogenous distribution of radioactivity throughout the brain. Following administration of increasing doses of the selective glycine transporter subtype 1 (GlyT1) 2-chloro-N—[(S)-[(2S)-1-methyl-2-piperidinyl](phenyl)methyl]-3-(trifluoromethyl)benzamide, a dose-dependent decrease of compound A uptake in the brain was observed.

### 1.3 PET Data Analysis

The PET data were analysed by using tracer kinetic modeling techniques to derive estimates of the tissue delivery (K1) and partition coefficient (PET volume of distribution—Vd). An input function representing the concentration of unchanged radiotracer in plasma was generated using discrete measures of the total plasma radioactivity and HPLC measures determining the parent radiotracer fraction. A generic tracer kinetic model (DEPICT) which estimates the tissues impulse response function was fitted to each of the individual tissue time activity curves to derive the appropriate parameters for each region (K1,Vd) (R. N. Gunn, S. R. Gunn, V. J. Cunningham. Cereb Blood Flow Metab. 2001, 21(6), 635-52; R. N. Gunn, S. R. Gunn, F. E. Turkheimer, J. A. Aston, V. J. Cunningham. J Cereb Blood Flow Metab. 2002, 22(12), 1425-39). Extraction fraction (K1) was stable across brain regions at 0.06 min⁻¹ and was not altered by pretreatment of the selective glycine transporter subtype 1 (GlyT1) 2-chloro-N—[(S)-[(2S)-1-methyl-2-piperidinyl](phenyl)methyl]-3-(trifluoromethyl)benzamide. The volume of distribution (Vd) varied from 5.6 in mid-brain to 3 in cortical regions with lowest uptake observed in the olfactory bulbs (Vd=2). Following the high dose (0.5 mg/kg) of the selective glycine transporter subtype 1 (GlyT1) 2-chloro-N—[(S)-[(2S)-1-methyl-2-piperidinyl](phenyl)methyl]-3-(trifluoromethyl)benzamide, Vd was reduced in all brain regions to that observed in olfactory bulbs, indicating near complete GlyT1 saturation. Analysis of the change in compound A derived Vd following increasing doses of the selective glycine transporter subtype 1 (GlyT1) 2-chloro-N—[(S)-[(2S)-1-methyl-2-piperidinyl](phenyl)methyl]-3-(trifluoromethyl)benzamide provided an intravenous ED₅₀ of 0.0225 mg/kg.

1-35. (canceled)

36. A compound of formula (I) or a salt thereof: 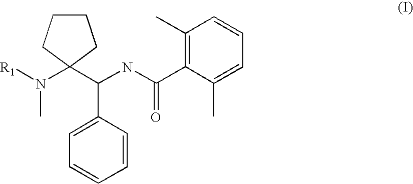
wherein R₁ is ³H or a C₁₋₆alkyl group having a radionuclide selected from the group consisting of ³H, ¹¹C, ¹⁴C, ¹³N, ¹⁵O, ⁷⁶Br, ¹⁸F, ¹²³I, ¹²⁵I, ¹³¹I, ⁷⁵Br, ⁷⁶Br, ⁷⁷Br and ⁸²Br.

37. A compound as claimed in claim 36 wherein R₁ is a C₁₋₆alkyl group having a radionuclide which is selected from the group consisting of ³H, ¹¹C, ¹⁴C, ¹³N, ¹⁵O, ⁷⁶Br, ¹⁸F, ¹²³I, ¹²⁵I, ¹³¹I, ⁷⁵Br, ⁷⁶Br, ⁷⁷Br and ⁸²Br.

38. A compound as claimed in claim 37, wherein R₁ is a C₁₋₆alkyl group having a ¹¹C carbon in said C₁₋₆alkyl group.

39. A compound as claimed in claim 36 which is [¹¹C—N-methyl]-N-[[1-(dimethylamino)cyclopentyl](phenyl)methyl]-2,6-dimethylbenzamide or a pharmaceutically acceptable salt thereof.

40. A method for labelling GlyT1 in a human which comprises administering to a patient an effective amount of a compound as defined in claim 36.

41. A method for diagnostic imaging of GlyT1 which comprises administering to a patient an effective amount of a compound as defined in claim 36.

42. A method for diagnostic imaging of tissues expressing GlyT1 which comprises administering to a human an effective amount of a compound as defined in claim 36.

43. A method for diagnostic imaging of GlyT1 in the brain of a human, which comprises administering an effective amount of a compound as defined in claim 36.

44. A compound as defined in claim 36 for use as a PET ligand or a SPECT ligand.

45. A pharmaceutical composition comprising a compound as claimed in claim 36 and at least one pharmaceutically acceptable carrier, diluent or excipient.

46. A pharmaceutical composition according to claim 45 comprising [¹¹C—N-methyl]-N-[[1-(dimethylamino)cyclopentyl](phenyl)methyl]-2,6-dimethylbenzamide or a pharmaceutically acceptable salt and at least one pharmaceutically acceptable carrier, diluent or excipient.
